(19) United States
(12) Patent Application Publication
(10) Pub. No.: US 2012/0277531 A1
(43) Pub. Date: Nov. 1, 2012

(54) Bend sensor

(76) Inventors: Beat Krattiger, Beringen (CH); Sebastian Wagner, Bretten (DE); Jerome Carrard, Sehaffhausen (CH); Sina Kraemer, Spiesen-Elversberg (DE)

(21) Appl. No.: 13/457,663

(22) Filed: Apr. 27, 2012

(30) Foreign Application Priority Data
Apr. 28, 2011 (DE) 10 2011 017 704.3

(51) Int. Cl. A61B 1/005

(52) U.S. Cl. 600/117

(57) ABSTRACT

A bend sensor for measuring a deflection of a technical or medical instrument, consisting of an elongated body of electrically insulating polymer material, with a longitudinal axis and with fibers of an electrically conducting polymer material that are embedded in the body. The fibers are disposed essentially parallel to the longitudinal axis and at a distance from one another in the polymer body. A measuring unit is connected with the fibers and is suited for evaluating the modification of the electrical resistances of the fibers as a measurement of the deflection of the body from the longitudinal axis.

### CROSS-REFERENCE TO RELATED APPLICATIONS

The present application claims priority of German patent application No. 10 2011 017 704.3 filed on Apr. 28, 2011.

### FIELD OF THE INVENTION

The invention relates to a bend sensor for measuring a deflection, consisting of an elongated body of electrically insulating polymer material with fibers embedded in the body that consist of an electrically conducting polymer material. The invention further relates to a medical and/or endoscopic instrument and a medical bracket with a bend sensor of the aforementioned type.

### BACKGROUND OF THE INVENTION

Bend sensors are used in many fields to determine the deflection of an object from its longitudinal axis, and frequently use electrically conductive material in order to make the direction or strength of the deflection measurable by a modification of resistance, capacity or inductivity. The bendable or deflectable objects are, for example, technical or medical instruments. Whenever two portions of an object constitute a variable angle with one another, bend sensors can be used to measure the bend.

In medical technology and medical or technical endoscopy, bend sensors are also used to determine the position and deflection of flexible or bendable instruments. In particular with flexible endoscopes, it has become customary to configure the distal end in such a way that it can be diverted by a hand control or by motor drive. In order to be able to guide the instrument with precision in tight spaces and body cavities, the deflection of the instrument insertion part, in particular of the distal end, should be recorded as precisely as possible. In the process, it is necessary that the end sensors being used measure the deflection along an X or Y axis. Because the instruments often have a small diameter and in addition already incorporate light conductors, image conductors, working channels, Bowden cables or the like, the bend sensors and their contacts and the necessary electric lines can occupy only limited space.

A bend sensor for use in joy sticks is known from U.S. Pat. No. 6,201,468 B1, with an elastic and electrically insulating base body into which a central strand and several surrounding strands of conductive rubber are admitted. Between the surrounding strands and the central strand there is a gap in each case, which is successively closed up only by bending the sensor. The contact surface between the surrounding and central strand and the electrical resistance measured thereby correspond with the sensor's direction and degree of curvature.

The disadvantage of this known bend sensor is that between the strands, gaps must remain, causing a greater sensor and requiring especially precise manufacturing with a smaller structural shape. The signal generated by the contact surface also fails to precisely reproduce the bend of the sensor, especially at great bend angles.

From U.S. Pat. No. 5,728,044 A, a flexible rod for insertion into working channels of flexible medical instruments is known, on whose surface a number of bend sensors are fastened in order to determine a deflection of the medical instrument and thus of the rod on the basis of the electric signals generated by the sensors.

This structure has the disadvantage that several sensors must be used in order to record movements in different spatial directions. For each sensor, separate electric lines are necessary, which also demand space. Also, the flat bend sensors used here are only suited to measure bends in one direction; the sensor cannot be bent in the direction of its narrow longitudinal side. They are also less flexible than the rod on which they are mounted, a fact that leads to erroneous measurement signals. In addition, this known rod is not protected against torsion, which adversely affects the measurement of the deflection. Additional torsion sensors are necessary to take possible twisting of the rod into account.

### SUMMARY OF THE INVENTION

It is consequently one object of the invention to provide an improved bend sensor that is simple to manufacture and makes precise measurement possible. It is another object of the invention to provide a medical and/or endoscopic instrument or a medical bracket with an improved bend sensor, so that a deflection can be recorded precisely.

The terms curvature, bend, angling and deflection may be used in alternation and designate the bend of the sensor, of the particular object on which the sensor is mounted, from its longitudinal axis. It is understood that also the difference between two deflected or arched conditions can be designated in this manner.

According to the invention, these objects are achieved through the features of a bend sensor for measuring deflection. Advantageous refinements are also disclosed.

An inventive bend sensor may comprise an elongated body made of electrically insulating polymer material into which fibers of electrically conductive polymer material are embedded. The fibers are disposed essentially parallel to one another and at a distance from one another in the body. The intervals between the fibers are filled by insulating polymer material. The bend sensor preferably has a cross-section that is approximately round or else square or can have a different shape that at least has a similar expanse along its axis of symmetry and thus allows a flexible and uniform bending of the sensor in many directions without preference for one direction on the basis of the geometric shape.

By determining the modification of the electrical resistances of two fibers with the help of a measuring unit that is connected with the fibers, it is possible to easily and reliably record a deflection of the sensor from its longitudinal axis in the plane in which both fibers are situated. The modification of the electrical resistances of the fibers here is approximately proportional to the degree of deflection and can be measured precisely. The polymer of the body and the polymer of the fibers can be an elastomer, for example a silicon or the like. The flexibility of the sensor body is preferably comparable with that of the fibers. The fibers are separated from one another by the body and do not touch one another.

Such an inventive bend sensor with polymer body and completely or partly embedded fibers can, in addition, easily be produced by co-extrusion and in any desired length. The cross-section of the fibers here can assume any desired form and for instance can be round or oval or can have the shape of a circle segment.

As a result of the simple and cost-effective configuration of the inventive bend sensor, a number of applications are possible. Thus, it can also be used as a redundant sensor to a highly precise bend sensor in robotics, and there can ensure additional security. Use in biomechanics is also thinkable in order to measure and reproduce the movement of living bodies.

In a preferred embodiment of the invention, the inventive bend sensor comprises at least four fibers, which are peripherally set apart from one another by about 90 degrees. With four fibers mounted in this manner it is possible to record a deflection both along an X-axis perpendicular to a longitudinal axis of the bend sensor and a Y-axis orthogonal to it, in two contrary directions in each case. Of course, all other bends, which are made up in their direction of an X- and a Y-portion, are also correctly measured by it. It is not necessary to use several sensors in order to record all these directions. The bend sensor, because of its inventive configuration, can also correctly measure curvatures with a large bending angle of more than 180 degrees.

According to another advantageous embodiment, means are also provided for stiffening the sensor that restrict or prevent a rotation of the bend sensor around its longitudinal axis. These can be any means that completely or nearly completely prevent torsion, but do not simultaneously impair the bending of the sensor body.

Advantageously for this purpose, the bend sensor is enclosed along its longitudinal axis, at least partly, by a braided hose that is contiguous on its body. The braided hose, according to the invention, can consist of a polyamide, for example nylon fibers, of glass, carbon or polyaramide and can be mounted on the body or cemented onto the body. The inventive braided hose can also be a braided layer, which is immediately mounted directly on the sensor by co-extrusion during manufacture and does not constitute a separate part.

It is possible, by means of the braided hose, to successfully prevent the bend sensor from being rotated around its longitudinal axis. This makes it superfluous to use expensive torsion sensors or the like, which record any undesired rotation in order to allow correction of the measuring signal. Thus, with a simple, economical step, it is possible to ensure correct functioning of the bend sensor. The braided hose, in addition, has the characteristic of protecting the sensor body from mechanical stress and/or of insulating it electrically from outside in cases where no electrical connection with other components is desired.

In a preferred version, the diameter of the bend sensor over its entire length is essentially smaller than one millimeter. Because of the special design of the bend sensor, it is possible to produce it in such small dimensions that it can comfortably be integrated into various medical and/or endoscopic instruments.

Only at this small diameter is it possible to use the bend sensor in flexible endoscopes, for example, where only little space is available in the shaft because of the many other components already present such as glass fibers or working channels. In medical technology in particular, the instruments may be inserted through narrow body openings, so that only a few square millimeters are available in the shaft cross-section.

According to another preferred embodiment, electrical lines contact the fibers in the area of the fiber ends. Said lines produce an electrical connection of the fibers among themselves and with the measuring unit in order to record the resistance change in the fibers as a measure of the deflection.

Here, in each case two of the fibers and the electric lines together with the measuring unit preferably form an electric switch, which is designated as a Wheatstone bridge and is known to the specialist. The variable electrical resistances here are formed by the fibers. The necessary reference resistances are, for example, housed in the measuring unit. Thus it is possible to detect a deflection of the bend sensor from its longitudinal axis. For the desired recording of deflections along the X- and Y-axes perpendicular to the longitudinal axis of the sensor, three switches of this kind are combined and the fibers are correspondingly electrically contacted. By offloading the reference resistances into a measuring unit, space can be saved in the sensor and in the apparatus in which the sensor is used.

In an additional advantageous embodiment of the bend sensor, the fibers are situated at least partly on the surface of the body. In this arrangement it is especially easy to contact the fibers electrically because they are not completely electrically insulated from outside by the polymer body.

According to another advantageous embodiment, the electric lines are at least partly embedded in the electrically insulating polymer material of the body. This now has the advantage that the lines are fed within the body and thus do not enlarge the diameter of the sensor. An especially compact structure thereby becomes possible.

The electric lines can be, for instance, wires that are conducted through the body and are each preferably connected at the fiber ends corresponding with the fibers.

Still more advantageously, the electric lines can include additional fibers embedded in the body and made of the conductive polymer material. These are electrically insulated for the most part by the body from the fibers used to record the deflection, and are electrically switched with them only at the fiber ends. According to the invention, such a sensor body with all necessary polymer fibers can be co-extruded in one piece and thus produced especially simply.

The electric connection at the fiber ends can advantageously be produced by wires or micro-nails, which are inserted into the fibers and, corresponding to the switch, connect the various fibers with one another. Various other types of contacting are also possible. If the fibers are situated partly on the surface of the body, the contacting can occur by way of the outside of the sensor.

In another especially suitable embodiment, the body is provided along its longitudinal axis with a channel, which can run centered through the body for example, such that the electric lines at least partly are conducted through the channel. In this manner, also after production of the sensor body, the lines can still be newly laid simply and compactly.

An especially simple contacting, also adapted to the inventive small diameter of the bend sensor, occurs according to an additional configuration by flex prints. The term “flex print” designates flexible circuit boards with printed conductor paths. These circuit boards are made, for example, of a synthetic material and are easily shapable in order to completely or partly enclose the bend sensor at the fiber ends. Contacts provided on the flex prints, corresponding to the switching, are directly connected with such fibers, which are situated partly on the surface of the body, but otherwise are embedded in the sensor body. Parts of the flex prints can also be guided through the canal to the proximal end of the sensor, where they are connected to the measuring unit with corresponding feeder lines.

In an inventive refinement of the bend sensor, the fibers and/or the flex prints are provided with indentations, into which protrusions of the respective other components, flex print or fiber, engage in order to ensure a secure contacting and to avoid slippage, and in addition in order to allow the flex prints to be as closely contiguous on the bend sensor as possible.

In another advantageous configuration, on the outside of the sensor a shrink hose surrounds the bend sensor over its entire length or in part in order to hold all components of the bend sensor such as sensor body, braided hose and electrical lines in the correct position and to avoid any slippage even during a deflection of the bend sensor. There can also be two or more shrink hose parts, which in particular are each mounted on the ends of the sensor. This constitutes a space-saving method for holding the components securely together. In order to be able to verify the correct contacting in simple manner during manufacture, the shrink hose is preferably of transparent construction.

Because of its compact structure and the possibly small dimensions, the inventive bend sensor is especially suited for use in medical and/or endoscopic instruments, for example flexible endoscopes. An inventive instrument comprises an insertion part, with at least one flexibly configured portion, so that the bend sensor is disposed in the insertion part in the area of this flexible portion in order to measure a deflection of the insertion part with the help of the bend sensor.

The bend sensor can advantageously be employed and can allow precise control of the shaft deflection in other instruments as well, which because of their requirements comprise a shaft with small diameter such as for example flexible gripping forceps, catheters or other medical instruments. Several flexible portions can also be provided, of course, on the insertion part, each equipped with a bend sensor.

According to another advantageous configuration, the bend sensor is thus completely enclosed by the insertion part. In this manner the outer structure of the instrument's insertion part can remain identical while including for example various hoses, braided nets and Bowden cables. Space must merely be foreseen inside the insertion part for the bend sensor, which is possible because of its inventive small dimensions. This allows simple modular addition of the sensor in existing instruments without the need for expensive reconstruction of the instruments. The sensor is installed simply and in protected manner in the shaft. The electric lines are thus preferably fed by the bend sensor through the insertion part to the proximal end of the instrument, where they are connected with the measuring unit.

In another advantageous configuration of an inventive medical and/or endoscopic instrument, a motor unit is in connection with the flexible portion of the instrument in order to cause deflection of the insertion part. The measuring unit and motor unit thus are connected with one another via signal lines to transmit a signal as a measurement of the deflection.

This now has the advantage that a power-driven deflection of the flexible portion can be measured and that the signals generated by the measuring unit can be used by feedback to the motor unit to allow better control of the power-driven deflection. The deflected position of the medical and/or endoscopic instrument is thereby recorded precisely and provides information as to whether the power-driven deflection must be corrected. This constitutes a considerable improvement of the entire instrument, because precise working with the insertion part is made possible, a factor of great significance especially in medical technology.

Precisely the same advantages accrue from using the inventive bend sensor in a medical bracket in which at least one jointly configured portion is foreseen. Thus the bend sensor, according to the invention, is disposed in this portion in order to measure a deflection of the bracket. Here too, feedback becomes possible concerning the actual deflection and position of the bracket, allowing the user to align the bracket reliably, in particular while using motors. Several jointed portions with several bend sensors can also be foreseen, of course. Use in medical robotics is also conceivable, where bend sensors are especially important for recording the alignment and movement. Because the inventive bend sensor can be produced especially easily and cost-effectively, a greater number of sensors can also be foreseen without problems.

The features cited above and those yet to be explained hereinafter can be applied not only in the specifically indicated combination, but also in other combinations or individually, without departing from the framework of the present invention.

The described invention may be distinguished by an elongated flexible bend sensor of electrically insulating synthetic material into which elongated fibers of electrically conductive plastic are inserted. The fibers run approximately parallel to the longitudinal axis of the sensor. A bending of the sensor leads to a modification of the electrical resistances in the fibers that is recorded by a measuring apparatus. It is electrically connected with the fibers of the sensor for this purpose. The electrical switching of the fibers with the components of the measuring apparatus corresponds advantageously to a Wheatstone bridge. The inventive bend sensor is distinguished by a compact and simple structure, such that a bending is recorded reliably and securely. Further means for stiffening the sensor can be foreseen, which make the sensor dimensionally stable against rotation around the longitudinal axis.

Embodiments of the invention are more closely depicted in the drawings and are explained in greater detail in the following description.

### BRIEF DESCRIPTION OF THE DRAWINGS

FIG. 1 shows a sketch of the inventive bend sensor with a measuring unit.

FIG. 2 shows the bend sensor with braided hose.

FIG. 3 shows the bend sensor according to a first embodiment.

FIG. 4 shows the bend sensor according to a second embodiment.

FIG. 5 shows the bend sensor according to a third embodiment.

FIG. 6 shows the bend sensor with shrink hose.

FIG. 7 shows the endoscopic instrument with bend sensor.

FIG. 8 shows the endoscopic instrument with bend sensor in the cross-section along the line A-A from FIG. 7.

FIG. 9 shows a sketch of the bend sensor in connection with a measuring unit and a motor unit.

FIG. 10 shows a medical bracket with bend sensor.

FIG. 11 shows a bend sensor according to an additional embodiment.

### DETAILED DESCRIPTION OF THE INVENTION

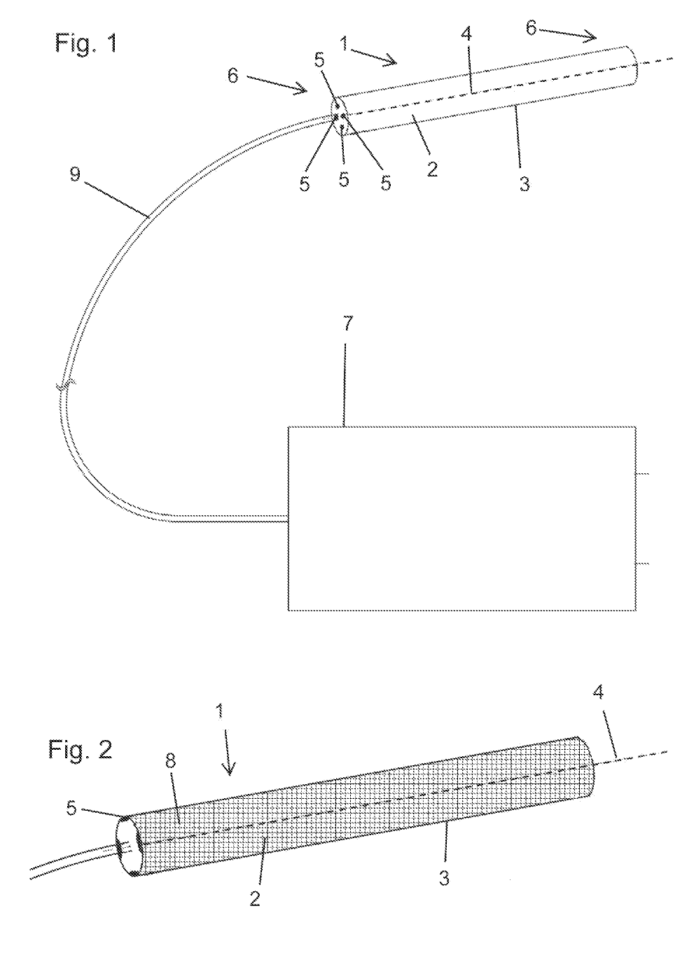

FIG. 1 shows schematically an inventive bend sensor 1 with a body 2 of electrically insulating polymer and four fibers 5 that are embedded in it and consist of electrically conducting polymer that are displaced peripherally from one another by about 90 degrees and disposed at a distance from one another. In the examples described hereinafter, the body consists of a silicon, and the fibers consist of an electrically conducting silicon-elastomer mixture. The electrical conductivity of the synthetic material is achieved, for example, by the mixture of nanoparticles of an electrically conducting material such as silver or graphite. The fibers 5 are contacted in the area of the fiber ends 6 by electric lines 9 that connect the bend sensor 1 with a measuring unit 7. Shown by way of example in FIG. 1 is just one of the electric lines 9.

If the bend sensor 1 is curved and thus deflected from its longitudinal axis 4, then the electrical resistance in the conducting polymer fibers 5 changes with the degree of curvature. This change is recorded in the measuring unit 7. This is later depicted still more precisely. Thus two fibers 5 opposite one another allow the determination of the curvature in a plane, while the two other fibers 5 allow the determination of the curvature in a plane perpendicular thereto. The actual curvature of the bend sensor 1 is determined by the combination of these two measurements. In this way, according to the invention, curvatures can be recorded in all directions correctly, simply and securely.

The bend sensor 1 depicted in FIG. 2 is surrounded along its longitudinal axis 4 by a braided hose 8, which is connected with the surface 3 of the body 2. In particular, a net-shaped braided hose of nylon or another material is saturated with silicon or polyurethane resin and applied to the body 2. In this way, rotation of the body 2 around its longitudinal axis is restricted or prevented, which could falsify the measurements of the deflection. The fibers 5 thus preferably run parallel to the longitudinal axis 4 of the body 2 of the bend sensor 1.

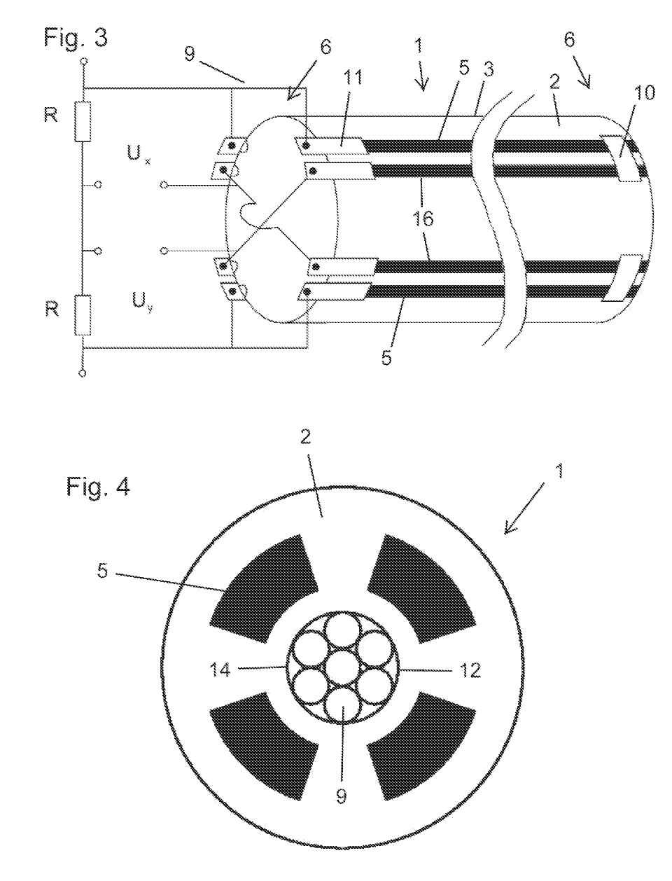

FIG. 3 shows the bend sensor according to a first embodiment, in which the four fibers 5 are disposed on the external surface 3 of the body 2. This allows a direct connection of the contacts 11 of the electric lines 9 with the fiber ends 6 on the outside of the body 2. As illustrated here, the electric lines 9 include four additional conducting polymer fibers 16, which are embedded in the body 2 in addition to the fibers 5 and run essentially parallel to the latter. In this manner the bend sensor, with the fibers 5, which form the variable resistances for measuring the deflection, and the additional fibers 16 for the electric line, can be co-extruded in one piece and thus can be produced especially quickly and simply and in any desired length. No lines are required to be conducted outside along the body 2 of the bend sensor 1, thus further reducing the necessary diameter of the bend sensor 1.

The electric lines 9 for connection with the measuring unit 7 attach exclusively on one end of the sensor body 2, shown here at the left. This has the advantage that all lines 9 can be directed in one direction away from the sensor 1 toward the measuring unit 7. Thus, particularly during use in an endoscope, all lines can easily be directed from the proximal end of the bend sensor 1 to the proximal end of the endoscope and to the measuring unit 7. At the other end, here shown at the right, it is necessary merely to secure a connection between the measuring fibers 5 and the additional fibers 16. This is possible by various methods known to the specialist, for example with micro-clamps made of metal, which are stuck into the polymer material and thus create a mechanically and electrically secure connection. Especially advantageous, however, is a connection via flex prints 10, because they are especially flat in configuration and can be applied narrowly around the sensor body 2. The lead tracks foreseen on the flex prints 10 then provide the electrical connection between the various fibers 5 and 16 of the bend sensor 1.

The illustration also shows the wiring of the fibers 9 and of the additional fibers 16 with the reference resistances R to a Wheatstone bridge. Modification of the electrical resistances of the fibers 5 with respect to the reference resistances R, as is caused by a bending of the bend sensor 1 in the electrically conducting fibers 5, leads to a modification of a signal voltage Ux or Uy, which corresponds to a deflection of the bend sensor 1 in an X- or Y-plane. The signal voltages Ux or Uy give information on the measurement of the deflection from the longitudinal axis 4. In addition, they provide the X- and Y-components of the deflection. The reference resistances can be positioned, for example, in the measuring unit 7, which also records and evaluates the signal voltages. The reference resistances can be equal or different in quantity, and thus the same resistances can serve as reference for both directions or else different reference resistances can be foreseen for the X- and Y-measurement.

The additional fibers 16 can also contribute to the measurement signal by their own resistance modification during bending.

By means of appropriate electrical wiring of the conducting fibers 5 and 16, it is also possible to produce an inventive bend sensor with only two additional polymer fibers 16, that is, with a total of six conducting fibers.

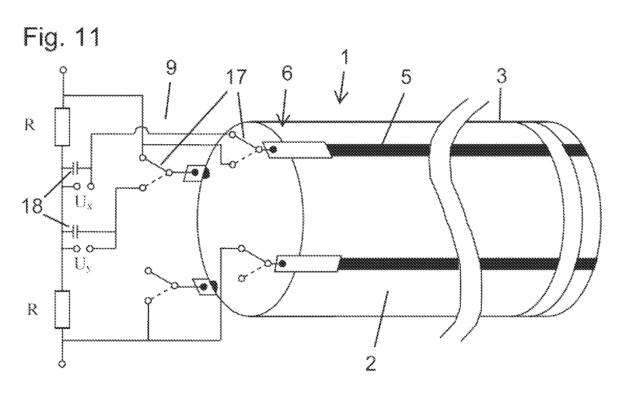

In an additional embodiment of the bend sensor 1, shown in FIG. 11, only four conducting fibers 5 are foreseen, which serve at one point to measure the deflection from the longitudinal axis 4 by their resistance which varies while bending, and at one point for electrical connection with the electric feeder lines 9 or as part of these feeder lines. For this purpose, the sensor 1 is connected in each case with switches 17 on the contacting end (shown at left) to the fiber ends 6, preferably with electronically synchronized semiconductor switches, which are also interconnected with one another. With the switch 17 in a first position, the current flows for example through those fibers 5 that serve to measure the deflection along the X-axis. The electrical connection of these measuring fibers with the feeder lines 9 then occurs via at least one fiber that is not used in this position for the measurement and that is associated with recording the deflection in the Y-direction.

With the switch 17 in a second position, here shown in broken lines, the resistance modification of the fibers foreseen for measuring the Y-deflection is analogously recorded and at least one fiber, which is associated with the measurement in the X-direction, is used as electric line 9. By permanent rapid switching between the first and second positions, both direction components X and Y of the deflection can be measured nearly simultaneously with the help of condensers 18.

At the end of the bend sensor 1 opposite the switches 17, the fibers 5 are electrically connected with one another, in simple manner as described in the other embodiments, in order to complete the switching.

The deflection can be recorded by appropriate switching as a separate voltage Ux and Uy, in each case for the X- and Y-component of the bending, or else measured as a voltage U, which during permanent switching corresponds in the first position to the voltage Ux and in the second position to the voltage Uy.

Owing to this especially simple structure of the bend sensor 1, the number of necessary fibers 5 is reduced, simplifying production.

FIG. 4 shows the cross-section of a bend sensor 1 according to another embodiment. Here the polymer fibers 5 are completely surrounded by the insulating body 2. The sensor 1 here has a round cross-section, allowing a light, uniform deflection from the longitudinal axis in all directions. The electric lines 9, which are connected on the fiber ends (not shown) with the fibers 5, are at least partly, in the form of wires, directed in a canal 12 that runs almost centrally along the longitudinal axis 4 through the bend sensor 1. In this manner the electric lines 9 do not enlarge the cross-section of the bend sensor 1. The connection of the electric lines 9 with the fiber ends occurs, for example, because the wires 9 pierce through the body 2 and the fibers 5 depending on the desired electrical switching. Because materials, over time, can lead to an aging or reshaping of the body 2, the canal 12 is equipped at each of its ends with a small tube 14 for reinforcement. The small tubes 14 prevent modification of the canal diameter from material fatigue or from material creep. These small tubes can be equipped with an insulation to ensure electrical insulation from the electric lines 9.

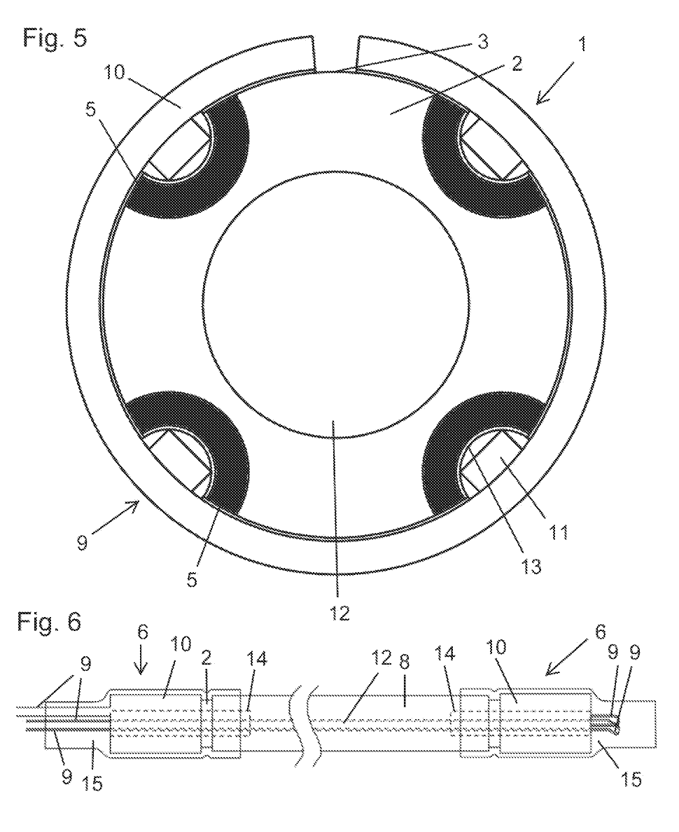

The bend sensor 1 illustrated in cross-section in FIG. 5, according to an additional embodiment, again includes a body 2 with a central canal 12 extending lengthwise through which a part of the electrical lines 9 can be fed. The conducting polymer fibers 5 here are situated partly on the surface of the body 2 and are contacted by flex prints 10 applied externally. The flex prints 10 are reshaped corresponding to the diameter of the body 2 and surround the body 2 tightly to avoid unnecessarily enlarging the diameter of the bend sensor 1. The flex prints 10 comprise contacts 11 in the form of protrusions, which engage in recesses 13 in the conducting fibers 5. It is also possible to equip the fibers 5, at least in the area of the fiber ends 6, with such protrusions, which conversely engage in recesses in the contacts 11. A combination of both is also possible. In any case, secure contacting is ensured and slippage of the contacts is prevented.

FIG. 6 shows a lateral depiction of the bend sensor 1. The body 2 is surrounded over its length by a braided hose 8 in the form of a braided layer co-extruded with the sensor. In the area of the fiber ends 6, the bend sensor, as described in the preceding embodiment, is contacted by flex prints 10. The other electric lines 9, which serve to connect the electrically conducting polymer fibers 5 with the measuring unit 7, which is not shown here, run through the central canal 12 from the end of the bend sensor 1, shown here at the right, to the left end of the bend sensor 1 and further to the measuring unit 7. In the illustrated example, the other electric lines 9 are continuations of the flex prints 10. The canal 12 is reinforced at either of its ends with a small tube 14. To ensure a secure grip by the flex prints 10, the sensor in the area of its ends is further equipped with a shrink hose 15, which firmly surrounds the body 2, the braided net 8 and the flex prints 10.

A secure arrangement is thereby provided, which even under difficult external conditions delivers reliable information for deflecting the sensor 1.

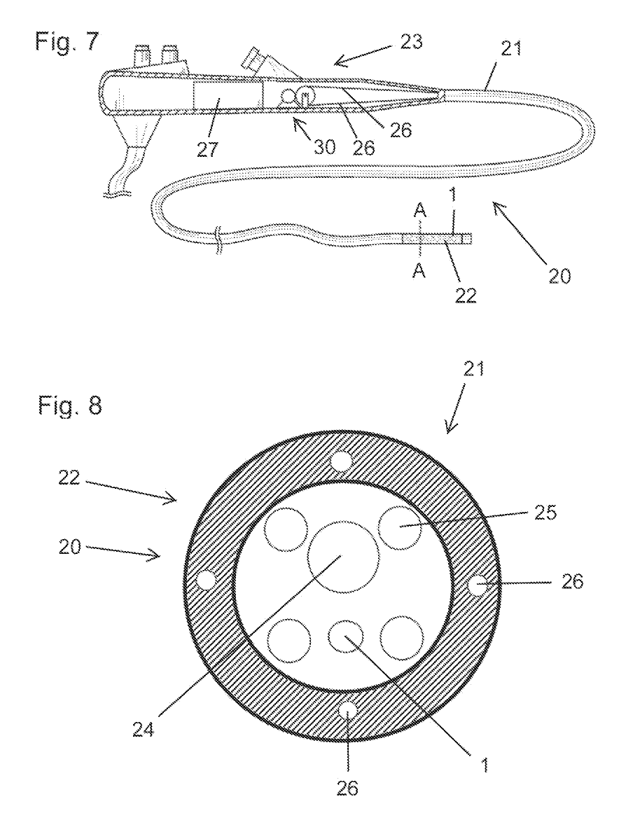

The endoscopic instrument illustrated in FIG. 7 is a flexible endoscope 20 and includes an insertion part 21 with a flexible portion 22, as well as a proximal housing 23. In this example the flexible portion 22 is disposed on the distal end of the insertion part 21. It is also possible, however, that one or more flexible portions can be foreseen, distributed over the insertion part. The Bowden cables 26 run from the inside of the housing 23 through the insertion part 21 to the flexible portion 22. Because of the movement of a mechanism 30 in the housing 23, the flexible portion 22 is deflected by means of the Bowden cables 26. This can be accomplished by hand by actuating an adjusting wheel, which is not illustrated, mounted on the housing 23, or else electrically by a motor unit 27. Here the Bowden cables 26 are connected with the motor unit 27 by the mechanism 30.

The flexible endoscope 20 also includes a bend sensor 1 according to one of the examples hitherto described, which is positioned in the area of the flexible portion 22. Given the presence of several flexible portions, it makes sense to equip each portion with its own bend sensor. If the flexible portion 22 is now deflected, the deflection is recorded by the bend sensor 1 and a corresponding signal is transmitted onward to the measuring unit 7, which is not illustrated here. The measuring unit 7, for this purpose, can be located outside the endoscopic instrument 20 or else can be integrated in the instrument 20, for example in the housing 23.

FIG. 8 shows a cross-section through the insertion part 21 of the flexible endoscope 20 in the area of the flexible portion 22 along the line A-A in FIG. 7.

The insertion part 21 of this instrument 20 comprises in its cross-section an image conductor 24, which transmits a distally received light signal onward in the proximal direction, along with light conductor bundles 25, which serve to transmit illuminating light onward from the proximal to the distal end. The Bowden cables 26 are shown in the outer area of the cross-section. To ensure uniform illumination of the object that is to be observed, several light conductor bundles 25 are commonly foreseen in the cross-section. In medical technology in particular, the diameter of the flexible endoscope shafts preferably amounts to only a few millimeters, so that only little space is available in the insertion part for additional components. The inventive bend sensor 1, which may be configured as shown in one of the aforementioned examples, may be disposed between the other components, image conductor 24 and light conductor 25, in the insertion part 21 and may be completely surrounded by it. Because of its compact structure and its small diameter of less than one millimeter, integration into the insertion part 21 of the endoscope is possible. The space proximally from the bend sensor 1 can be used to house the electric lines 9 and to direct them to the proximal end of the instrument 20, which contributes to a compact total structure of the flexible endoscope 20.

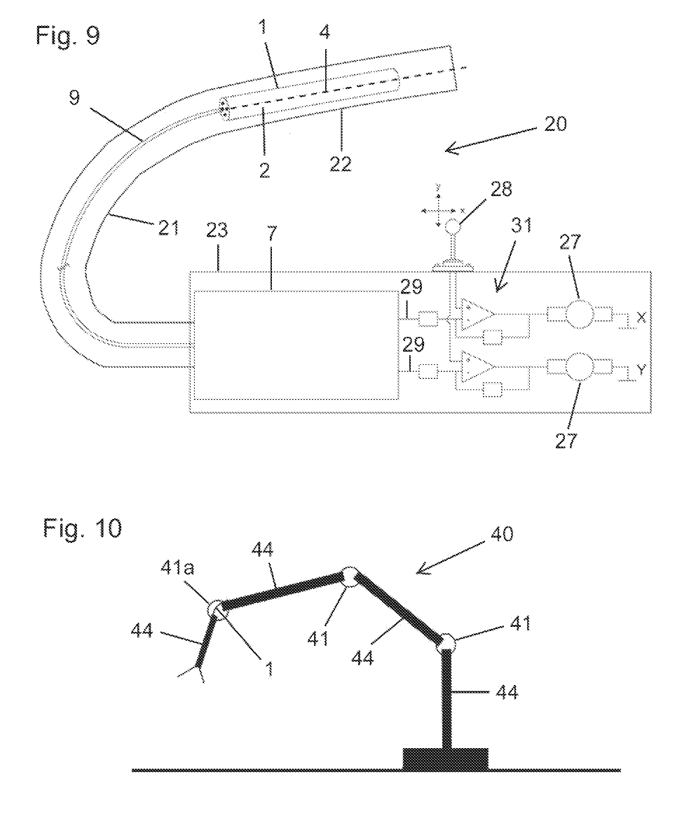

FIG. 9 clarifies the connection between the bend sensor 1 and the measuring unit 7 or motor units 27, as the sensor can be foreseen in a flexible endoscope 20. As previously described with reference to FIG. 7, the bend sensor 1 is situated in the flexible portion 22 of the insertion part 21 of the endoscope 20. The measuring unit 7 is now foreseen in the proximal housing 23. Signal lines 29 connect the measuring unit 7 with a control unit 31, which on the one hand is connected with a joystick 28 and on the other hand with the motor units 27. By actuating the joysticks 28, the motor units 27 are caused via the control unit 31 to move the Bowden cables 26 by the mechanism 30, which is not shown here. In this manner the flexible portion 22 of the insertion part 21 is deflected.

Because the bend sensor 1 is also deflected, it generates a signal that represents the actual bending of the portion 22 and that is transmitted to the measuring unit 7. The latter evaluates the signal and transmits an adapted control signal onward to the control unit 31. In this manner, there is an automatic compensation for mechanical insufficiencies, as occur in flexible endoscopes for example because of the presence of belt friction. Consequently, the actuation of the joystick 28 correlates exactly with movement of the flexible portion 22. Mechanical influences that arise are compensated during the application, because the bend sensor 1 continuously supplies precise information on the actual degree of deflection of the flexible portion 22 to the measuring unit 7.

An inventive bracket 40 is shown in FIG. 10. Such a medical bracket 40 serves, for example, to hold medical instruments in an operating room. It includes in this example three jointed portions 41, 41a, which allow the different parts 44 of the bracket to be deflected with respect to one another. In the jointed portion 41a, a bend sensor 1 is foreseen based on one of the aforementioned embodiments. It reliably records a deflection of the parts 44 of the bracket 40 that are connected together by the jointed portion. It is understood that any desired number of jointed portions 41, 41a can be provided with an inventive bend sensor 1 in order to record an alignment of the bracket 40 with overall precision.

1. A bend sensor for measuring a deflection, comprising an elongated body made of electrically insulating polymer material, with a longitudinal axis, with fibers of an electrically conducting polymer material embedded in the body, such that the fibers are disposed essentially parallel to the longitudinal axis and at a distance from one another in the polymer body, wherein a measuring unit is provided that is connected with the fibers and is suited for determining the modification of the electric resistances of the fibers as a measurement of the deflection of the body from the longitudinal axis.

2. The bend sensor according to claim 1, wherein at least four fibers are disposed in the body peripherally offset by about 90 degrees from one another.

3. The bend sensor according to claim 1, wherein means for stiffening the sensor are provided that restrict rotation of the bend sensor around its longitudinal axis.

4. The bend sensor according to claim 3, wherein the body is surrounded along its longitudinal axis by a braided hose contiguous with the body.

5. The bend sensor according to claim 1, wherein its diameter over its entire length is smaller than 1 mm.

6. The bend sensor according to claim 1, wherein electric lines contact the fibers in the area of the fiber ends.

7. The bend sensor according to claim 1, wherein the fibers are at least partly contiguous with the surface of the body.

8. The bend sensor according to claim 1, wherein two of the fibers and the electric lines together with the measuring unit form an electrical switch in the form of a Wheatstone bridge.

9. The bend sensor according to claim 1, wherein the electric lines are at least partly embedded in the electrically insulating polymer material of the body.

10. The bend sensor according to claim 9, wherein the electric lines include additional fibers of the electrically conducting polymer material that are embedded in the body.

11. The bend sensor according to claim 1, wherein the body comprises a canal that is, in particular, central along its longitudinal axis and through which at least a part of the electrical lines are fed.

12. The bend sensor according to claim 1, wherein the electric lines include flex prints with electric contacts that are contiguous with the body and contact the fibers.

13. The bend sensor according to claim 12, wherein the fibers and/or the flex prints comprise recesses in which protrusions of the respective other component, flex print or fiber, engage.

14. The bend sensor according to claim 1, wherein a shrink hose at least partly surrounds the bend sensor.

15. The bend sensor according to claim 1, wherein it is integrated into a medical or endoscopic instrument with an insertion part that comprises at least one flexible portion, and wherein the bend sensor is disposed in the area of the flexible portion in the insertion part in order to measure a deflection of the insertion part.

16. The bend sensor according to claim 15, wherein the bend sensor is completely surrounded by the insertion part.

17. The bend sensor according to claim 15, wherein at least one motor unit is operationally linked with the flexible portion of the instrument in order to cause a deflection of the insertion part, such that the measuring unit and the motor unit are connected with one another via signal lines to transmit a signal as a measurement of the deflection.

18. The bend sensor according to claim 1, wherein it is integrated in a medical bracket with at least one jointly configured portion, such that the bend sensor is disposed in this portion in order to measure a deflection of the bracket.
